(19) United States
(12) Patent Application Publication
(10) Pub. No.: US 2018/0268417 A1
(43) Pub. Date: Sep. 20, 2018

(54) Methods and Systems for a FHIR Interface for Customer Relationship Management Systems

(71) Applicant: Michigan Health Information Network - MiHIN, East Lansing, MI (US)

(72) Inventors: Jeff Livesay, Pinckney, MI (US); Tim Pletcher, East Lansing, MI (US)

(21) Appl. No.: 15/982,875

(22) Filed: May 17, 2018

Related U.S. Application Data
(63) Continuation of application No. 15/461,904, filed on Mar. 17, 2017.
(60) Provisional application No. 62/310,882, filed on Mar. 21, 2016.

(51) Int. Cl. G06Q 30/00; G16H 10/60

(52) U.S. Cl. CPC G06Q 30/01; G16H 10/60

(57) ABSTRACT

An illustrative method includes receiving, from a requestor device, a request to read, write, edit, or delete a resource data. The request is formatted according to a Fast Healthcare Interoperability Resources (FHIR) standard and FHIR extensions embodying an healthcare provider directory (HPD) standard. The method further includes generating an application program interface (API) request configured for a customer relationship management (CRM) database to request the read, write, edit, or delete of the resource data. The method further includes sending the API request to the CRM database. The CRM database is configured to receive a plurality of request types. The method further includes receiving, from the CRM database, data responsive to the API request. The method further includes generating data responsive to the request from the data responsive to the API request. The method further includes sending the data responsive to the request.

### CROSS-REFERENCE TO RELATED APPLICATION

This application is a Continuation Application of U.S. patent application Ser. No. 15/461,904 filed Mar. 17, 2017, which claims priority to provisional U.S. Patent Application No. 62/310,882, filed on Mar. 21, 2016, each of which is incorporated herein by reference in its entirety.

### BACKGROUND

Record keeping is widely practiced throughout the world. Record keeping can be used in many various industries and for many various purposes. For example, record keeping may be utilized in industries such as health care, banking, finance, retail, services, or any other industry. Records can take various forms including, but not limited to, financial statements, receipts, medical records, legal documents, insurance records, employment records, payroll records, confidential records, e-mail, website content, forms of identification, etc. Records may also be kept or stored in various ways. For example, records may be stored physically in files or electronically on cloud storages, document management systems, secure servers, hard drives, etc.

In just one example, records are utilized in the health care industry for various purposes. For example, a physician may keep records relating to appointments for consumers, consumer demographic information, and/or records of consumer visits including vitals, diagnoses, and treatments. Such records may be used to generate billing information for the consumer, an insurance provider of the consumer, or another third party payee such as a government agency. The physician may also keep such billing information as records.

Records are important to many individuals, businesses, and governments. Accordingly, records are the subject of much attention with regards to how records are identified, classified, prioritized, stored, secured, archived, preserved, retrieved, tracked, and destroyed. Decisions regarding records are made based on many different factors including applicable laws, company policies, expected future utilization of a record, type of record, importance/value of record, etc.

### SUMMARY

An illustrative method according to a set of instructions stored on the memory of a computing device includes receiving, from a requestor device, a request to read, write, edit, or delete a resource data. The request is formatted according to a Fast Healthcare Interoperability Resources (FHIR) standard and FHIR extensions embodying an healthcare provider directory (HPD) standard. The method further includes generating an application program interface (API) request configured for a customer relationship management (CRM) database to request the read, write, edit, or delete of the resource data. The method further includes sending the API request to the CRM database. The CRM database is configured to receive a plurality of request types. The method further includes receiving, from the CRM database, data responsive to the API request. The method further includes generating data responsive to the request from the data responsive to the API request. The data responsive to the request is formatted according to the FHIR standard and the FHIR extensions embodying the HPD standard. The method further includes sending, to the requestor device, the data responsive to the request.

An illustrative non-transitory computer readable medium having instructions stored thereon that, upon execution by a computing device, cause the computing device to perform operations, wherein the instructions include instructions to receive, from a requestor device, a request to read, write, edit, or delete a resource data. The request is formatted according to a Fast Healthcare Interoperability Resources (FHIR) standard and FHIR extensions embodying an healthcare provider directory (HPD) standard. The instructions further include instructions to generate an application program interface (API) request configured for a customer relationship management (CRM) database to request the read, write, edit, or delete of the resource data. The instructions further include instructions to send the API request to the CRM database. The CRM database is configured to receive a plurality of request types. The instructions further include instructions to receive, from the CRM database, data responsive to the API request. The instructions further include instructions to generate data responsive to the request from the data responsive to the API request. The data responsive to the request is formatted according to the FHIR standard and the FHIR extensions embodying the HPD standard. The instructions further include instructions to send, to the requestor device, the data responsive to the request.

An illustrative apparatus includes a memory, a processor coupled to the memory, and a first set of instructions stored on the memory and configured to be executed by the processor. The processor is configured to receive, from a requestor device, a request to read, write, edit, or delete a resource data. The request is formatted according to a Fast Healthcare Interoperability Resources (FHIR) standard and FHIR extensions embodying an healthcare provider directory (HPD) standard. The processor is further configured to generate an application program interface (API) request configured for a customer relationship management (CRM) database to request the read, write, edit, or delete of the resource data. The processor is further configured to send the API request to the CRM database. The CRM database is configured to receive a plurality of request types. The processor is further configured to receive, from the CRM database, data responsive to the API request. The processor is further configured to generate data responsive to the request from the data responsive to the API request. The data responsive to the request is formatted according to the FHIR standard and the FHIR extensions embodying the HPD standard. The processor is further configured to send, to the requestor device, the data responsive to the request.

### BRIEF DESCRIPTION OF THE DRAWINGS

Illustrative embodiments will hereafter be described with reference to the accompanying drawings.

FIG. 1 is a block diagram illustrating an example computer in accordance with an illustrative embodiment.

FIG. 2 is a diagram illustrating an example Fast Healthcare Interoperability Resources (FHIR) Application Program Interface (API) in accordance with an illustrative embodiment.

FIG. 3 is a flow diagram illustrating a method for utilizing a Fast Healthcare Interoperability Resources (FHIR) Application Program Interface (API) in accordance with an illustrative embodiment.

FIG. 4 is a flow diagram illustrating a method for reading a secure email address from a database in accordance with an illustrative embodiment.

FIG. 5 is a flow diagram illustrating a method for editing a contract resource in a database in accordance with an illustrative embodiment.

FIG. 6 is a flow diagram illustrating a method for writing fields to a resource in a database in accordance with an illustrative embodiment.

FIG. 7 is a flow diagram illustrating a method for writing a new resource in a database in accordance with an illustrative embodiment.

### DETAILED DESCRIPTION

Disclosed herein are systems and methods for a Fast Healthcare Interoperability Resources (FHIR) Interface (or Application Program Interface (API)) for Customer Relationship Management (CRM) systems, which can be realized in software, hardware, or a combination thereof. FHIR is an interoperability standard for the transfer of clinical and administrative data between software applications and various healthcare entities, databases, etc. FHIR is part of the Health Level 7 (HL7) standards propagated by the standards organization Health Level Seven International. The FHIR standard version DSTU2 is incorporated herein by reference. An FHIR interface methods and systems disclosed herein provide a server front end and API for CRM systems such as HighRise™, Sugar™, Goldmine™, Salesforce.com™, Netsuite™, Microsoft Dynamics™ CRM, Hubspot™, and Gotoassist™. The FHIR interface methods and systems disclosed herein can also provide a server front end and API for provider relationship management systems such as those used by healthcare providers. This interface can allow various systems, databases, etc. to be communicated with through a single API whether or not the system currently uses the FHIR standard such that communications with these systems can be conducted as if these systems did use the FHIR standard. In other words, the systems and methods disclosed herein allow a FHIR standard system to interoperate with systems that are or are not utilizing FHIR standards. Other advantages and features of the FHIR interface are disclosed herein.

The systems and methods disclosed herein provide a way for accessing information stored as part of a CRM system or any other type of database so that the information can be leveraged through the FHIR API to accomplish various goals. For example, the FHIR API may be used to access or verify a healthcare provider's secure email account address. In another example, the FHIR API may be used to look up a doctor or other healthcare provider on the Internet, such as on WebMD™, and then using the FHIR API to determine communication means such as a secure email address for that doctor. Conversely, a consumer may also have a secure email account that is stored in a directory that can be looked up by a healthcare provider in order to contact that consumer. In addition to end users such as consumers and healthcare providers, directories and other databases may also be able to communicate with each other through the FHIR API. For example, a provider database may be able to communicate through the FHIR API with another database to update or learn about consumer preferences that are stored in other databases such as consent to clinical trials, immunization history-forecasts. In another example, a provider database may communicate through the FHIR API to do quality reporting to a state agency database.

Advantageously, the FHIR API disclosed herein can communicate with databases that already use the FHIR standard, as well as databases that do not, such as a CRM system. If a system that previously has not used FHIR, the systems and methods disclosed herein can be updated once such systems start to use the FHIR standard. Such a system solves a technical problem of interoperability between databases before some databases adopt the same interoperability standard, but others have already adopted an interoperability standard. Adopting new interoperability and communication standards takes considerable time and effort, and is an ongoing and incremental process. Additionally, some systems may not ever adopt certain interoperability standards, such as the FHIR standard. Thus, in order to leverage the information stored in a CRM and other systems that may not have adopted an interoperability standard such as FHIR, the disclosed API can be used to help end users and various databases, directories, and CRMs communicate, whether or not any of them have adopted the FHIR standard. Finally, databases, directories, servers, etc. that are already conformed to the FHIR standard can communicate with each other and a CRM utilizing the methods and systems herein.

Also disclosed herein are extensions to the FHIR standard which allow for further capabilities of a FHIR API for CRM. For example, certain extensions can supplement the resources of FHIR with additional resources. As just one example, resources related to Integrating the Healthcare Enterprise (IHE) Electronic Health Records (EHR) standards for healthcare provider directories (HPD) (collectively, IHE/EHR HPD) so that the FHIR API can support other use cases such as Electronic Service Information (ESI), taxonomies, and membership. In some embodiments, these extensions may add new resources beyond what is included in FHIR DSTU2. In other embodiments, the extensions may add new data fields for existing resources in FHIR DSTU2.

In an illustrative embodiment, the systems and methods disclosed herein include extensions to the FHIR standard that fully support Integrating the Healthcare Enterprise (IHE) Electronic Health Records (EHR) standards for healthcare provider directories (HPD) (collectively, IHE/EHR HPD)that allow the CRM system disclosed herein with the FHIR API to serve as a provider directory that is fully conformant with the IHE/EHR HPD standard. The IHE/EHR HPD extensions to FHIR in the disclosed herein include support in FHIR for Electronic Service Information (ESI), taxonomies, and membership. Furthermore, in this embodiment, IHE/EHR HPD extensions allow the CRM system with the FHIR API to function as a Software Development Kit (SDK) that other applications can invoke to utilize the functionality of the FHIR API so that the CRM functioning as Provider Directory can support use cases in healthcare including care coordination (such as managing consumer-provider attribution information), and also valuable use cases for quality measurement and reporting.

In another illustrative embodiment, the system includes full FHIR API support for the CRM system to function as an electronic consent management system (ECMS) such as the ECMS described in the ECMS Specification v1.1., published by the CIO Forum in Michigan on Mar. 26, 2015, and incorporated herein by reference. This embodiment uses the specification only as a reference model, and instead of using Query By Parameter (QBP) as called for in the specification, the system uses FHIR contracts resources to support the specification for ECMS thereby providing a more lightweight and efficient way to manage consumer consent and a faster, lower cost way to interoperate with other consent management systems using FHIR instead of the more heavyweight, more time-consuming, and more expensive QBP protocol.

In another illustrative embodiment, the system includes FHIR API support that enables a CRM system to operate as a Consumer Directory which stores consumer healthcare preferences and the FHIR API makes these consumer preferences available to other applications for use cases such as Care Coordination use cases like managing consumer-provider attribution, admission-discharge-transfer notifications, and medication reconciliation. The embodiment disclosed herein also supports communication of other consumer preferences such as consent to clinical trial, immunization history-forecast, share information with consumer and other use cases related to consumer preferences. A consumer as described herein refers to any person who could consume healthcare services. When consuming healthcare services, a consumer may be referred to as a patient.

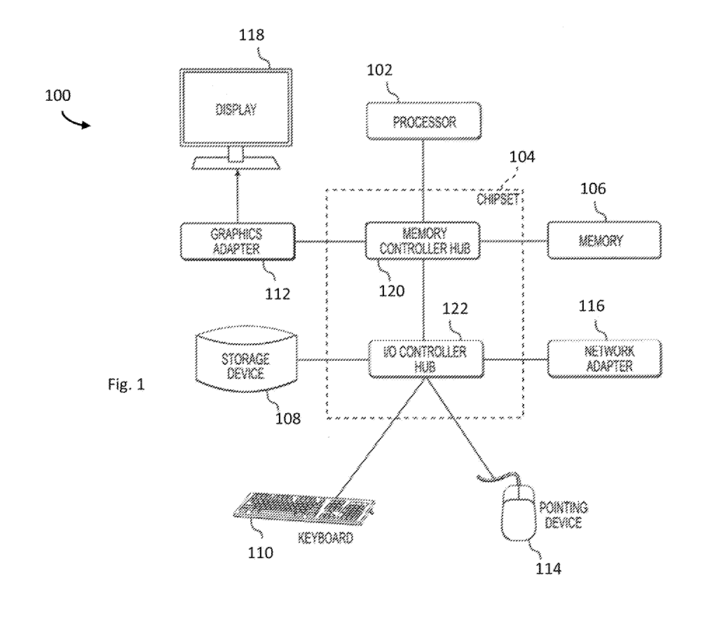

FIG. 1 is a block diagram illustrating an example computer 100 in accordance with an illustrative embodiment. In alternative embodiments, fewer, additional, and/or different components may be present. The computer 100 may be any computing machine, such as a tablet, smart phone, laptop computer, desktop computer, server, remote client device, gaming device, smart television device, wearable computer, or any combination thereof. The computer 100 includes at least one processor 102 coupled to a chipset 104. The chipset 104 includes a memory controller hub 120 and an input/output (I/O) controller hub 122. A memory 106 and a graphics adapter 112 are coupled to the memory controller hub 120, and a display 118 is coupled to the graphics adapter 112. A storage device 108, keyboard 110, pointing device 114, and network adapter 116 are coupled to the I/O controller hub 122. Other embodiments of the computer 100 may have different architectures.

The storage device 108 is a non-transitory computer-readable storage medium such as a hard drive, compact disk read-only memory (CD-ROM), DVD, or a solid-state memory device. The memory 106 holds instructions and data used by the processor 102. The pointing device 114 is a mouse, track ball, or other type of pointing device, and is used in combination with the keyboard 110 to input data into the computer 100. The pointing device 114 may also be a gaming system controller, or any type of device used to control the gaming system. For example, the pointing device 114 may be connected to a video or image capturing device that employs biometric scanning to detect a specific user. The specific user may employ motion or gestures to command the point device 114 to control various aspects of the computer 100.

The graphics adapter 112 displays images and other information on the display 118. The network adapter 116 couples the computer system 100 to one or more computer networks.

The computer 100 is adapted to execute computer program modules for providing functionality described herein. As used herein, the term module refers to computer program logic used to provide the specified functionality. Thus, a module can be implemented in hardware, firmware, and/or software. In one embodiment, program modules are stored on the storage device 108, loaded into the memory 106, and executed by the processor 102.

The types of computers used by the entities and processes disclosed herein can vary depending upon the embodiment and the processing power required by the entity. The computer 100 may be a mobile device, tablet, smartphone or any sort of computing element with the above-listed elements. For example, a data storage device, such as a hard disk, solid state memory or storage device, might be stored in a distributed database system comprising multiple blade servers working together to provide the functionality described herein. The computers can lack some of the components described above, such as keyboards 110, graphics adapters 112, and displays 118.

The computer 100 may act as a server. The computer 100 may be clustered with other computer 100 devices to create the server. The various computer 100 devices that constitute the server may communicate with each other over a network.

As would be appreciated by one of ordinary skill in the art, the embodiments disclosed herein may be implemented on any system, network architecture, configuration, device, machine, or apparatus, and is not to be construed as being limited to any specific configuration, network, or systems, even though an example system is shown and described with respect to FIG. 1. The embodiments herein may be suitably implemented on conventional computing devices, for example, computer workstations, on Internet based applications, on optical computing devices, neural computers, biological computers, molecular computing devices, and other devices. As may be appreciated by those skilled in the art, the present invention, in short, may be implemented on any system, automaton, and/or Turing machine.

An automaton is herein described as a mechanism that is relatively self-operating and designed to follow a predetermined sequence of operations or respond to encoded instructions. A Turing machine is herein described as an abstract expression of a computing device that may be realized or implemented on an infinite number of different physical computing devices. Examples of systems, automatons and/or Turing machines that may be utilized in performing the process of the present invention include, but are not limited to: electrical computers (for example, an International Business Machines (IBM) personal computer); neuro-computers (for example, one similar to the “General Purpose Neural Computer” described in U.S. Pat. No. 5,155,802, issued to Paul H. Mueller, on Oct. 13, 1992); molecular computers (for example, one similar to the “Molecular Automata Utilizing Single or Double-Strand Oligonucleotides” described in U.S. Pat. No. 5,804,373, issued to Allan Lee Schweiter et al., on Sep. 8, 1998); biological computers (for example, one similar to the biological computer presented by Ehud Shapiro, of the Computer Science and Applied Mathematics Department at the Weizman Institute of Science (Rehovot, Israel), at the Fifth International Meeting on DNA-Based Computers); and optical computers. For purposes of simplicity, such devices hereinafter are referred to as computers, as is commonly understood in the art. But, the embodiments disclosed herein are not limited being applied to such devices and may be accomplished upon any system or collection of systems capable of providing the features and functions identified herein.

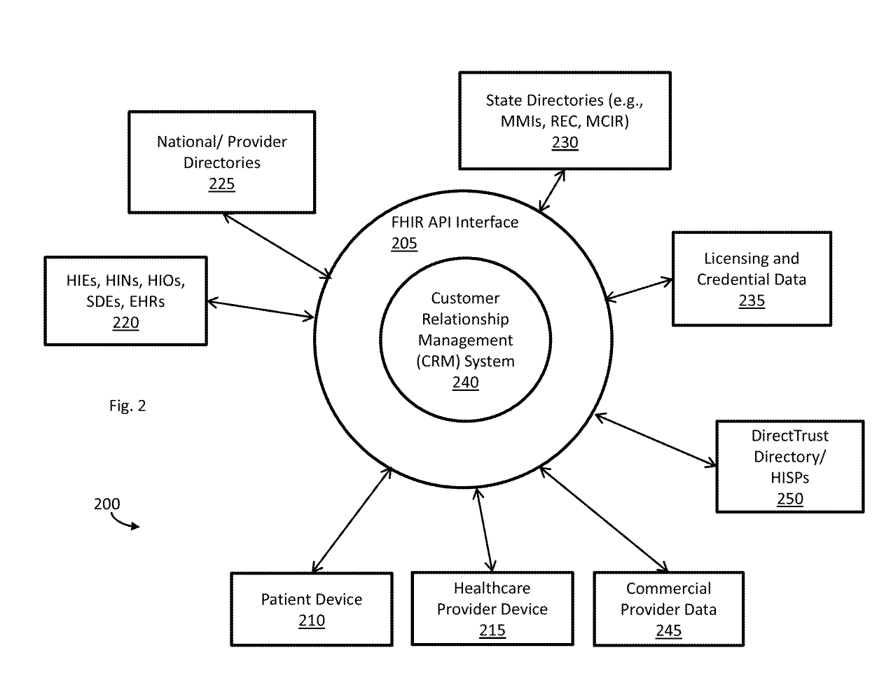

The methods described herein and with respect to FIGS. 2-7 may be performed partially or wholly on a processor, such as the one described above with regards to computer 100.

Certain of the devices shown in FIG. 1 include a computing system. The computing system includes a processor (CPU) and a system bus that couples various system components including a system memory such as read only memory (ROM) and random access memory (RAM), to the processor. The aspects disclosed herein may be suitably implemented on conventional computing devices, for example, computer workstations, on Internet based applications, on optical computing devices, neural computers, biological computers, molecular computing devices, and other devices. As may be appreciated by those skilled in the art, the aspects disclosed herein may be implemented on any system, automaton, and/or automated machine.

Other system memory may be available for use as well. The computing system may include more than one processor or a group or cluster of computing system networked together to provide greater processing capability. The system bus may be any of several types of bus structures including a memory bus or memory controller, a peripheral bus, and a local bus using any of a variety of bus architectures. A basic input/output (BIOS) stored in the ROM or the like, may provide basic routines that help to transfer information between elements within the computing system, such as during start-up. The computing system further includes data stores, which maintain a database according to known database management systems. The data stores may be embodied in many forms, such as a hard disk drive, a magnetic disk drive, an optical disk drive, tape drive, or another type of computer readable media which can store data that are accessible by the processor, such as magnetic cassettes, flash memory cards, digital versatile disks, cartridges, random access memories (RAMs) and, read only memory (ROM). The data stores may be connected to the system bus by a drive interface. The data stores provide nonvolatile storage of computer readable instructions, data structures, program modules and other data for the computing system.

To enable human (and in some instances, machine) user interaction, the computing system may include an input device, such as a microphone for speech and audio, a touch sensitive screen for gesture or graphical input, keyboard, mouse, motion input, motion detection, camera for video and photo input, and so forth. An output device can include one or more of a number of output mechanisms, such as a display screen, a printer, or speaker. In some instances, multimodal systems enable a user to provide multiple types of input to communicate with the computing system. A communication interface generally enables the computing device system to communicate with one or more other computing devices using various communication and network protocols.

Embodiments disclosed herein can be implemented in digital electronic circuitry, or in computer software, firmware, or hardware, including the herein disclosed structures and their equivalents. Some embodiments can be implemented as one or more computer programs, i.e., one or more modules of computer program instructions, encoded on a tangible computer storage medium for execution by one or more processors. A computer storage medium can be, or can be included in, a computer-readable storage device, a computer-readable storage substrate, or a random or serial access memory. The computer storage medium can also be, or can be included in, one or more separate tangible components or media such as multiple CDs, disks, or other storage devices. The computer storage medium does not include a transitory signal.

As used herein, the term processor encompasses all kinds of apparatus, devices, and machines for processing data, including by way of example a programmable processor, a computer, a system on a chip, or multiple ones, or combinations, of the foregoing. The processor can include special purpose logic circuitry, e.g., an FPGA (field programmable gate array) or an ASIC (application-specific integrated circuit). The processor also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, module, engine, software, software application, script, or code) can be written in any form of programming language, including compiled or interpreted languages, declarative or procedural languages, and the program can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

To provide for interaction with an individual, the herein disclosed embodiments can be implemented using an interactive display, such as a graphical user interface (GUI). Such GUI's may include interactive features such as pop-up or pull-down menus or lists, selection tabs, and other features that can receive human inputs.

The computing system disclosed herein can include clients and servers. A client and server are generally remote from each other and typically interact through a communications network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

FIG. 2 is a diagram illustrating an example Fast Healthcare Interoperability Resources (FHIR) Application Program Interface (API) in accordance with an illustrative embodiment. In alternative embodiments, fewer, additional, and/or different elements may be present. The system 200 of FIG. 2 includes a Customer Relationship Management (CRM) system 240 and a Fast Healthcare Interoperability Resource (FHIR) Application Program Interface (API) 205. The FHIR API 205 is wrapped around the CRM system 240 such that various directories, databases, etc., can communicate with the CRM system 240 through the FHIR API 205. As disclosed herein, directories, databases, etc. that are FHIR conformant or not may communicate with the CRM system 240 through the FHIR API. If a directory, database, etc. does not conform to FHIR, the FHIR API 205 may communicate with such a directory, database, etc. with another method of communication. As just one example, a RESTful API may be used.

FIG. 2 shows various directories, databases, etc. that can communicate with the CRM system 240 through the FHIR API 205. For example, the CRM system 240 through the FHIR API 205 may communicate with health information exchanges (HIEs), health insurance networks (HINs), health information organizations (HIOs), state designated entities (SDEs), and/or electronic health records (EHRs) 220. The CRM system 240 through the FHIR API 205 may also communicate with national and/or provider directories 225. The CRM system 240 through the FHIR API 205 may also communicate with state directories 230, such as MMIs, regional extension centers (RECs), and/or child immunization registries (CIRs). The CRM system 240 through the FHIR API 205 may also communicate with licensing and credential databases 235. The CRM system 240 through the FHIR API 205 may also communicate with a DirectTrust directory and/or health information service providers (HISPs) 250. The CRM system 240 through the FHIR API 205 may also communicate with commercial provider data 245. Additionally, the CRM system 240 through the FHIR API 205 may also communicate with end user devices such as a consumer device 210 and a healthcare provider device 215.

The various devices and directories, databases, etc. of FIG. 2 may communicate with each other and with the CRM system 240 through the FHIR API 205. Various ways in which these communications may be made are disclosed herein.

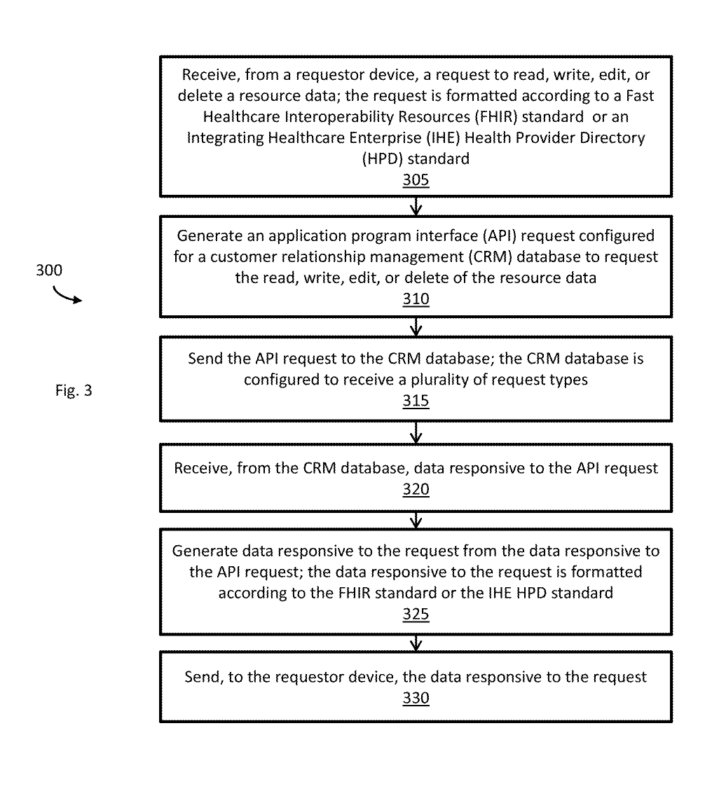

FIG. 3 is a flow diagram illustrating a method 300 for utilizing a Fast Healthcare Interoperability Resources (FHIR) Application Program Interface (API) in accordance with an illustrative embodiment. In alternative embodiments, fewer, additional, and/or different operations may be performed. Also, the use of a flow diagram is not meant to be limiting with respect to the order of operations performed. In an operation 305, the system receives, from a requestor device, a request to read, write, edit, or delete a resource data. The request is formatted according to a Fast Healthcare Interoperability Resources (FHIR) standard and/or the FHIR extensions embodying or incorporating the IHE/EHR HPD standard. The system may include a FHIR API that is wrapped around a customer relationship management (CRM) system as disclosed herein. In an alternative embodiment, if a directory, end user device, or other requestor device is not FHIR or IHE/EHR HPD conformant, the request may not be formatted according to the FHIR standard. The request may instead be formatted according to some other format or standard. As discussed herein, various embodiments may use a modified FHIR standard that includes extensions to the FHIR standard. Such extensions may or may not include resources and data fields associated with the IHE/EHR HPD standard. When the extensions are associated with the IHE/EHR HPD standard, the modified FHIR standard used by the API will conform to FHIR but will advantageously also include all or some of the capabilities of the IHE/EHR HPD standard.

Resources generally represent granular clinical concepts. The resources can be managed in isolation, or aggregated into complex documents. Such flexibility offers coherent solutions for interoperability. Each resource carries a master id. The master id identifies the resource permanently. Resources may refer to other resources by id knowing that this is a stable reference. Each resource type may have a URL which is derived from the id, the type, and the local base URL. Given one resource address, the address of any other resource may be automatically determined. Resources can contain references to other resources. Each resource supports the same list of transactions—read, write, edit, update, delete, etc. Another transaction type supported by resource types is the provision of a conformance statement which specifies what parts of the defined content model are supported by the system, and what other transactions or interactions are supported. If any of the other interactions are supported, the conformance interaction must be supported. (i.e. if the conformance interaction returns an error, no operations are supported).

The request received in the operation 305 may be related to a specific resource. The request is formatted according to a Fast Healthcare Interoperability Resources (FHIR) standard and the FHIR extensions embodying or incorporating the IHE/EHR HPD standard. In other words, the resource invoked by the request may be a resource defined by the FHIR standard or be formatted according to FHIR and be a FHIR extension embodying or incorporating something from the IHE/EHR HPD standard. By including at least some resources of both standards, a wider functionality can be achieved for the system. This can be accomplished by incorporating resources from the IHE/EHR HPD standard into the FHIR API functionality and capability. Such incorporation has many advantages that are discussed in greater detail herein.

Particular resources that may be read, written, edited, updated, deleted, etc. may include an organization resource. An organization resource may be a formally or informally recognized grouping of people or organizations formed for the purpose of achieving some form of collective action. An organization may include companies, institutions, corporations, departments, community groups, healthcare practice groups, etc. The organization resource may include data fields that may be read, written, edited, updated, deleted, etc., as disclosed herein. The data fields may include id, organization name, organization type, organization identifier or related objects, organization address, organization contact information, etc. Another resources are a practitioner, or a person who is directly or indirectly involved in the provisioning of healthcare. A practitioner may have some similar data fields to the organization, and may include fewer or other fields. For example, some data fields of a practitioner resource may include an active or non-active status field, provider name, provider gender, provider birthdate, qualifications, credentials, etc. Another resource is a bundle, which serves as a container for a collection of other resources. Another resource is a consumer, which may have data fields such as name, active or non-active status, contact information, address, gender, birthdate, contact information, consumer affiliations, etc. Another resource is a contract that may include data fields such as when a contract was issued, period of the contract, subject of the contract whose information is to be shared, type of information to be shared, actors (senders, recipients) in the sharing of information, signer, legal document reference (e.g. URL), etc. Advantageously, contract resources may be used to manage consent from consumers for information sharing between various organizations, directories, databases, etc., such as those shown in FIG. 2 and described above.

As disclosed herein, the number and types of resources and data fields for each resource may be different and various configurations are contemplated by the present disclosure. In addition, extensions to the FHIR DSTU2 standard are contemplated that add resources (e.g., resources from the IHE/EHR HPD standard) as well as extensions that add data fields to resources of the FHIR DSTU2 standard. For example, extensions to the organization resource may include additional data fields such as electronic services, taxonomies or organization specialties, and/or organization credentials or qualifications. A practitioner resource may also include taxonomies or electronic services extensions. Extensions may also be used to add resources beyond what is typical in the FHIR standard. For example, a membership resource may be added that denotes a formal relationship between a practitioner or organization and another organization in which the member participates or has participated. Data fields for a membership resource may include an id of the membership, electronic service information, membership type, organization reference, member reference, identifier by which a member is known to an organization, a period that defines effective dates of a membership, etc. Another added extension resource may include an electronic service resource with data fields such as name of the service, share services for which the service is a destination, content data types consumed by a service, networking protocol expected by a service, service's delivery address (e.g., Direct email address, internet protocol (IP) address, logical address), etc.

Another extension resource may be a service description resource, which wraps an electronic service resource and its hosting organization into an object (or resource itself) that contains adequate information for an end user to select a particular membership in which to participate as a member. The data fields for such a resource may include service descriptions, types of service, name of service, image to represent a service, reference to an organization offering the service, a reference to an electronic service that supports the service description, etc.

Another extension resource may be an active care relationship resource which is used to record members of a consumer's care team as reported to a care relationship service. The active care relationship service may include data fields such as administrator, consumer, provider, practice, period of time of active care, etc.

In an operation 310, the system generates an application program interface (API) request configured for a customer relationship management (CRM) database to request the read, write, edit, or delete of the resource data. In other words, the system, based on the request, generates the API call that is to be sent into the CRM to read, write, edit, or delete a resource or a data field of a resource. In an operation 315, the system sends the API request to the CRM database. The CRM database is configured to receive a plurality of request types, such as reads, writes, edits, deletes, etc. These request types may cause different actions or associations based on the type of request and the resource and data fields that changed, added, deleted, etc.

In an operation 320, the system receives, from the CRM database, data responsive to the API request. This data responsive to the API request is data sent from the CRM database in response to the API request that was sent to the CRM database in the operation 315, described above.

In an operation 325, the system generates data responsive to the request from the data responsive to the API request. The data responsive to the request is formatted according to the FHIR standard and/or the FHIR extensions embodying or incorporating the IHE/EHR HPD standard. Here, the FHIR API generates a response to the original request from the requestor device received in the operation 305, described above. In other words, the system prepares the response that is to be sent to the requestor device based on the data responsive to the API request received from the CRM. In an alternate embodiment, the format of the data responsive to the original request may be in a format other than FHIR or IHE/EHR HPD if the requestor device is not FHIR or IHE/EHR HPD conformant.

In an operation 330, the system sends, to the requestor device, the data responsive to the request. In alternative embodiments, the data responsive to the request may be sent to other devices than the requestor device, either instead of or in addition to the requestor device. Such an embodiment advantageously allows the system to, for example, update a resource in the CRM and, in sending data responsive to the request, update other databases that should also be update with the same or related information. For example, a consumer may sign a consent form to release medical records and a request may come in to update the consent contract resource for the consumer and/or the consumer resource. The system may also wish to update a healthcare provider that the contract was executed.

Accordingly, the system may send the data responsive to the request in the form of a confirmation that the contract was executed and properly saved in the CRM to both the requestor device that was used to execute the contract and to the healthcare providers that will releasing and receiving the consumer's health information. The system may have predefined rules for how to generate data responsive to requests (e.g., when to send a confirmation of an update or delete of a resource, when to send the actual resource as data responsive to the request). The system may also have predefined rules on where and who to send data responsive to requests to based on the type of request, the resource that is affected, etc.

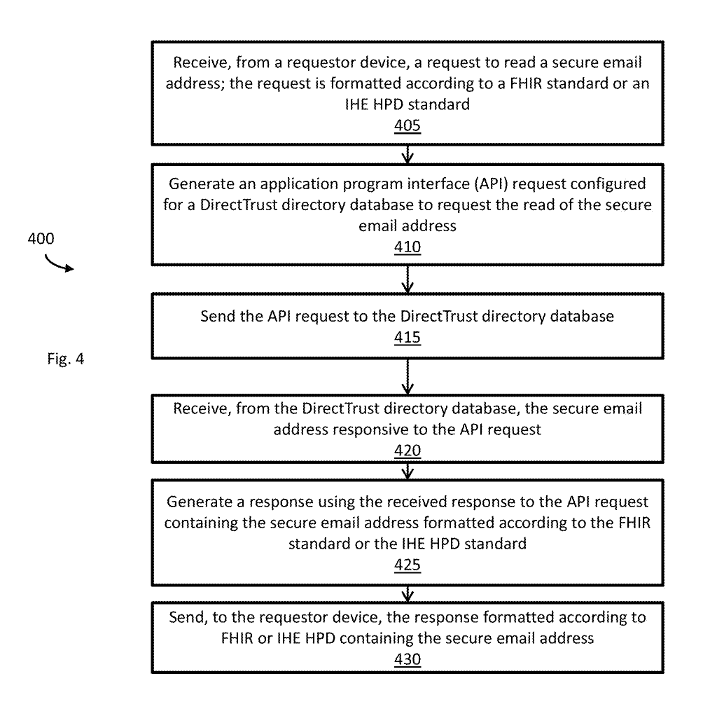

FIGS. 4-7 further describe additional illustrative embodiments for the methods and systems discussed above and shown in FIGS. 1-3. FIG. 4 is a flow diagram illustrating a method 400 for reading a secure email address from a database in accordance with an illustrative embodiment. In alternative embodiments, fewer, additional, and/or different operations may be performed. Also, the use of a flow diagram is not meant to be limiting with respect to the order of operations performed. In an operation 405, the system receives, from a requestor device, a request to read a secure email address. The request is formatted according to a FHIR standard and/or FHIR extensions embodying or incorporating an IHE/EHR HPD standard. As above, in alternative embodiments, the request may not be formatted according to FHIR or IHE/EHR HPD. In this embodiment, someone may be looking up how to contact a provider or consumer that has a secure email address. Accordingly, the secure email address can be requested and read from a directory. The response, ultimately discussed below with respect to an operation 430, will include the secure email address or otherwise include instructions for how to contact the party using a secure email address.

In an operation 410, the system generates an application program interface (API) request configured for a DirectTrust directory database to request the read of the secure email address. In an operation 415, the system sends the API request to the DirectTrust directory database. In an operation 420, the system receives, from the DirectTrust directory database, the secure email address responsive to the API request. In an operation 425, the system generates a response using the received response to the API request containing the secure email address formatted according to the FHIR standard and/or FHIR extensions embodying or incorporating the IHE/EHR HPD standard. In alternative embodiments, a response may be formatted differently. In an operation 430, the system sends, to the requestor device, the response formatted according to FHIR and/or FHIR extensions embodying or incorporating the IHE/EHR HPD standard containing the secure email address. Thus, a requestor device can receive a secure email address for a provider or consumer they are looking up.

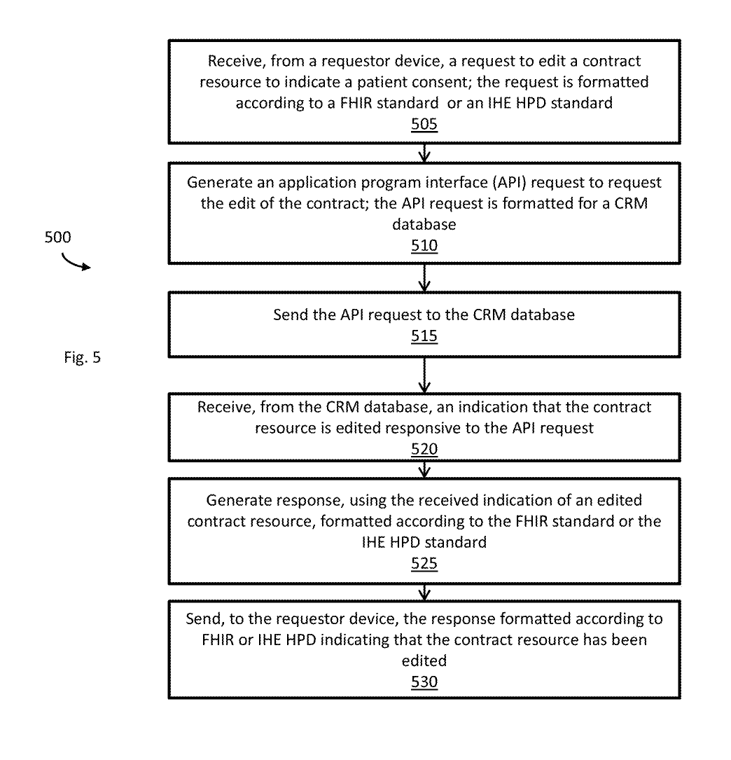

FIG. 5 is a flow diagram illustrating a method 500 for editing a contract resource in a database in accordance with an illustrative embodiment. In alternative embodiments, fewer, additional, and/or different operations may be performed. Also, the use of a flow diagram is not meant to be limiting with respect to the order of operations performed. In an operation 505, the system receives, from a requestor device, a request to edit a contract resource to indicate a consumer consent. The request is formatted according to a FHIR standard and/or FHIR extensions embodying or incorporating an IHE/EHR HPD standard. In alternative embodiments, the request may be formatted differently. Here, a requestor device is updating or creating a consent using the contract resource as disclosed herein. The requestor device may be a device on which the contract has been executed, or a database or directory in which the contract has been stored and subsequently wants to update a CRM to reflect the contract for consumer consent.

In an operation 510, the system generates an application program interface (API) request to request the edit of the contract. The API request is formatted for a CRM database. In an operation 515, the system sends the API request to the CRM database. In an operation 520, the system receives, from the CRM database, an indication that the contract resource is edited responsive to the API request. In an operation 525, the system generates a response, using the received indication of an edited contract resource, formatted according to the FHIR standard and/or the FHIR extensions embodying or incorporating the IHE/EHR HPD standard. In alternative embodiments, a response may be formatted differently. In an operation 530, the system sends, to the requestor device, the response formatted according to FHIR and/or the FHIR extensions embodying or incorporating the IHE HPD standard indicating that the contract resource has been edited.

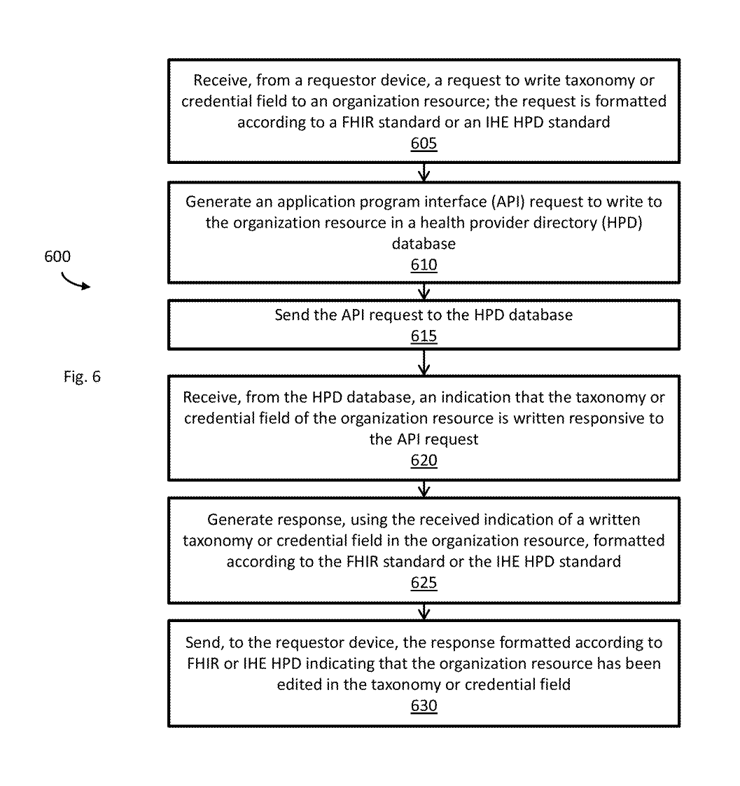

FIG. 6 is a flow diagram illustrating a method 600 for writing fields to a resource in a database in accordance with an illustrative embodiment. In alternative embodiments, fewer, additional, and/or different operations may be performed. Also, the use of a flow diagram is not meant to be limiting with respect to the order of operations performed. In an operation 605, the system receives, from a requestor device, a request to write to a taxonomy or credential data field to an organization resource. The request is formatted according to a FHIR standard and/or FHIR extensions embodying or incorporating an IHE/EHR HPD standard. In alternative embodiments, the request may be formatted differently. Here, a provider device or database storing information about providers, for example, may seek to update its credentials or taxonomy classifications in the CRM database.

In an operation 610, the system generates an application program interface (API) request to write to the organization resource in a health provider directory (HPD) database. In one embodiment, the HPD may be integrated into the CRM. In other embodiments, the HPD is separate from the CRM and the system generates an API call to the HPD database and a CRM. In another embodiment, the system generates only an API call to the HPD and does not generate an API call to the CRM. In this way, the FHIR API as disclosed herein can be used to facilitate communication between various databases, directories, etc. even where the CRM is not necessarily implicated.

In an operation 615, the system sends the API request to the HPD database. In an operation 620, the system receives, from the HPD database, an indication that the taxonomy or credential field of the organization resource is written responsive to the API request. In an operation 625, the system generates a response, using the received indication of a written taxonomy or credential field in the organization resource, formatted according to the FHIR standard and/or the FHIR extensions embodying or incorporating the IHE/EHR HPD standard. In alternative embodiments, a response may be formatted differently. In an operation 630, the system sends, to the requestor device, the response formatted according to FHIR and/or the FHIR extensions embodying or incorporating the IHE/EHR HPD standard indicating that the organization resource has been edited in the taxonomy or credential field. In this way, a taxonomy or credential field of an organization or provider may be updated.

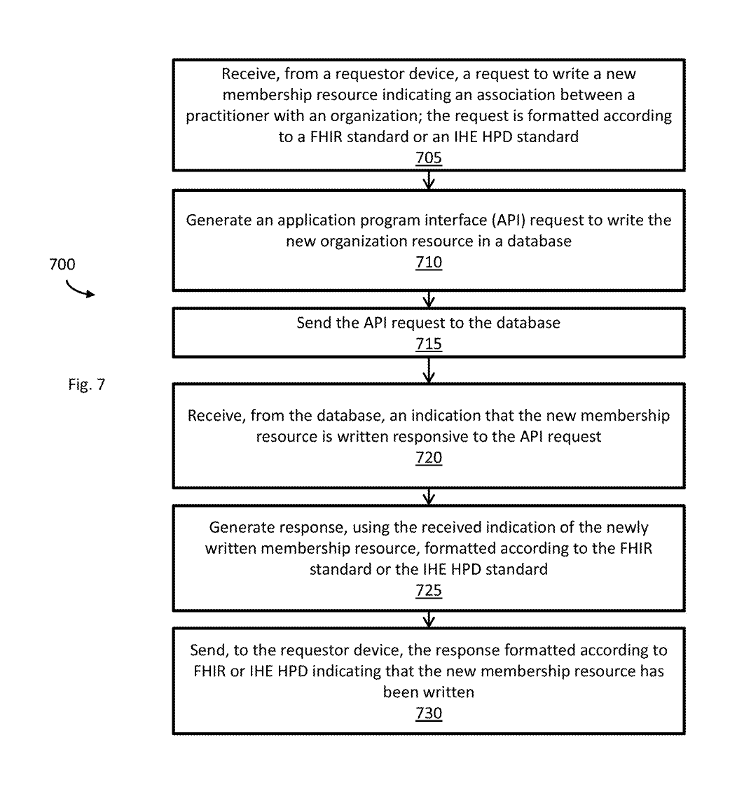

FIG. 7 is a flow diagram illustrating a method for 700 writing a new resource in a database in accordance with an illustrative embodiment. In alternative embodiments, fewer, additional, and/or different operations may be performed. Also, the use of a flow diagram is not meant to be limiting with respect to the order of operations performed. In an operation 705, the system receives, from a requestor device, a request to write a new membership resource indicating an association between a practitioner with an organization. The request is formatted according to a FHIR standard and/or FHIR extensions embodying or incorporating an IHE/EHR HPD standard. In alternative embodiments, the request may be formatted differently. Here, the request is to create a new membership resource that links a provider or organization to another organization as disclosed herein.

In an operation 710, the system generates an application program interface (API) request to write the new organization resource in a database. The database may be part of the CRM or separate from the CRM according to various embodiments. In an operation 715, the system sends the API request to the database. In an operation 720, the system receives, from the database, an indication that the new membership resource is written responsive to the API request. In an operation 725, the system generates a response, using the received indication of the newly written membership resource, formatted according to the FHIR standard and/or the FHIR extensions embodying or incorporating the IHE/EHR HPD standard. In alternative embodiments, a response may be formatted differently. In an operation 730, the system sends, to the requestor device, the response formatted according to FHIR or IHE/EHR HPD indicating that the new membership resource has been written.

In an illustrative embodiment, any of the operations described herein can be implemented at least in part as computer-readable instructions stored on a computer-readable medium or memory. Upon execution of the computer-readable instructions by a processor, the computer-readable instructions can cause a computing device to perform the operations.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method according to a set of instructions stored on the memory of a computing device, the method comprising:
receiving, from a requestor device, a request to read, write, edit, or delete a resource data, wherein the request is formatted according to a Fast Healthcare Interoperability Resources (FHIR) standard and FHIR extensions embodying a healthcare provider directory (HPD) standard;
generating an application program interface (API) request configured for a customer relationship management (CRM) database to request the read, write, edit, or delete of the resource data;
sending the API request to the CRM database, wherein the CRM database is configured to receive a plurality of request types;
receiving, from the CRM database, data responsive to the API request;
generating data responsive to the request from the data responsive to the API request, wherein the data responsive to the request is formatted according to the FHIR standard and the FHIR extensions embodying the HPD standard; and
sending, to the requestor device, the data responsive to the request.

2. The method of claim 1, wherein the requestor is a healthcare provider device or healthcare consumer device.

3. The method of claim 1, wherein the resource data is an application program interface (API).

4. The method of claim 1, wherein the resource data comprises a plurality of data field types.

5. The method of claim 4, wherein the plurality of data field types comprises at least one of an electronic service field, taxonomy field, or a qualification field.

6. The method of claim 4, further comprising editing or writing the resource data associated with a first data field type in response to the API request comprising data associated with a second data field type.

7. The method of claim 6, wherein the data associated with the second data field type comprises a secure e-mail address and the first data field type is an electronic service field.

8. The method of claim 1, wherein the resource data is associated with at least one of a plurality of types of resources.

9. The method of claim 8, wherein the plurality of types of resources comprises a membership resource indicating a formal relationship between a consumer, a practitioner, or first organization and a second organization in which the consumer, the practitioner, or the first organization has participated.

10. The method of claim 8, wherein the plurality of types of resources comprises an electronic service resource indicating information required to securely transit protected health information from one system to another electronically.

11. The method of claim 8, wherein the plurality of types of resources comprises a service description resource comprising an electronic service resource and an organization resource wrapped together into an object such that the request comprises a selection of a membership resource in which to participate as a member.

12. The method of claim 8, wherein the plurality of types of resources comprises an active care relationship resource indicating a member of a consumer's care team.

13. The method of claim 8, wherein the plurality of types of resources comprises a contract resource to manage electronic consent of a consumer.

14. The method of claim 1, further comprising authenticating or authorizing the request before receiving the data responsive to the API request.

15. The method of claim 1, wherein the CRM serves as a provider directory (PD) that is conformant with the HPD standard.

16. The method of claim 1, wherein the request comprises care coordination information of a consumer.

17. The method of claim 16, wherein the care coordination information comprises consumer-provider attribution information, admission-discharge-transfer notification information, health plan enrollment and population health participation, medication reconciliation information.

18. The method of claim 1, wherein the care coordination information comprises consumer preference information for storing personal health information (PHI), consent to clinical trial, advance directives, immunization history-forecast, programs or services in which a consumer is enrolled, family members or legal custodians, or managing relationships and communications between a provider and consumer.

19. The method of claim 1, wherein the HPD standard comprises an Integrating Healthcare Enterprise Electronic Health Record (IHE/EHR) HPD standard.

20. A non-transitory computer readable medium having instructions stored thereon that, upon execution by a computing device, cause the computing device to perform operations, wherein the instructions comprise:
instructions to receive, from a requestor device, a request to read, write, edit, or delete a resource data, wherein the request is formatted according to a Fast Healthcare Interoperability Resources (FHIR) standard and FHIR extensions embodying a healthcare provider directory (HPD) standard;
instructions to generate an application program interface (API) request configured for a customer relationship management (CRM) database to request the read, write, edit, or delete of the resource data;
instructions to send the API request to the CRM database, wherein the CRM database is configured to receive a plurality of request types;
instructions to receive, from the CRM database, data responsive to the API request;
instructions to generate data responsive to the request from the data responsive to the API request, wherein the data responsive to the request is formatted according to the FHIR standard and the FHIR extensions embodying the HPD standard; and
instructions to send, to the requestor device, the data responsive to the request.

21. An apparatus comprising:
a memory;
a processor coupled to the memory; and
a first set of instructions stored on the memory and configured to be executed by the processor, wherein the processor is configured to:
receive, from a requestor device, a request to read, write, edit, or delete a resource data, wherein the request is formatted according to a Fast Healthcare Interoperability Resources (FHIR) standard and FHIR extensions embodying a healthcare provider directory (HPD) standard;
generate an application program interface (API) request configured for a customer relationship management (CRM) database to request the read, write, edit, or delete of the resource data;
send the API request to the CRM database, wherein the CRM database is configured to receive a plurality of request types;
receive, from the CRM database, data responsive to the API request;
generate data responsive to the request from the data responsive to the API request, wherein the data responsive to the request is formatted according to the FHIR standard and the FHIR extensions embodying the HPD standard; and
send, to the requestor device, the data responsive to the request.
